(19) United States
(12) Patent Application Publication
(10) Pub. No.: US 2021/0268123 A1
(43) Pub. Date: Sep. 2, 2021

(54) DRUG FORMULATION FOR DERMAL DISEASES

(71) Applicant: MASTER DYNAMIC LIMITED, Hong Kong (CN)

(72) Inventors: Ka Wing CHENG, Hong Kong (CN); Wing Chi TANG, Hong Kong (CN); Koon Chung HUI, Hong Kong (CN)

(73) Assignee: MASTER DYNAMIC LIMITED, Hong Kong (CN)

(21) Appl. No.: 17/254,685

(22) PCT Filed: Jun. 21, 2019
(86) PCT No.: PCT/CN2019/092173

§ 371 Date: Dec. 21, 2020

(30) Foreign Application Priority Data
Jun. 22, 2018 (HK) 18108035.0

(51) Int. Cl. A61K 47/69; A61K 31/436; A61K 9/00; A61K 41/00

(52) U.S. Cl. CPC A61K 47/6923; A61K 41/0042; A61K 9/0014; A61K 31/436

(57) ABSTRACT

A conjugate comprising at least one calcineurin phosphatase inhibitor and a nanodiamond.

### TECHNICAL FIELD

The present invention relates to a topical product and formulation for the treatment of dermal disorders, in particular the present invention provides a formulation for the treatment of dermal diseases of subjects with decreased toxicity side effects.

### BACKGROUND OF THE INVENTION

There exist numerous dermal type diseases or disorders which cause an affliction to the skin or dermis of a subject. Such diseases can be communicable, curable, preventable, manageable or controllable. Such diseases can be highly noticeable on the skin of a subject, as well as in some cases not being visible

In addition to the inconvenience, and at times debilitating effects of such diseases, subjects afflicted with such diseases often suffer psychological effects by being uncomfortable or self-conscious in the presence of other persons, as well as be exposes to social rejection or at times ridicule.

As such, it is important that such diseases are effectively and efficiently treated, so as to at least render an affliction manageable or reduced to a manageable level, or where possible treated an eradicated.

To treat dermal diseases, depending upon the type of disease, a most typical method is to apply topical medications to affected areas. These topical medications can be in various forms, such as a cream, an ointment, a gel, a foam, a lotion, a spray or the like, all of which act as media or delivery agent to deliver the relevant active pharmaceutical ingredient or ingredients (API) of the drug product to the disease affected skin areas.

In other cases, depending upon the type of skin disorder, medicaments can be administered orally by way of a tablet, capsule, syrup or the like, or by way of intravenous (IV) delivery, which are also frequently used in combination with topical administration.

However, drug products and medicaments often have side effects, and although an API may have a suitable efficacy against a disorder to affliction, side effects from the usage of such drug products are also required to be considered in the use of all drug products, including dermal type applications,

As such, no matter how clinically effective a drug is, the dosage delivered to a subject must be controlled very carefully. When the prescribed or requisite dosage to treat an affliction is exceeded or a treatment regime exceeds the requisite amount drug to a subject, undesirable side effects can occur. Hence, if a treatment is not applied appropriately or regime exceeds the safe working dosage, a subject patient may suffer from side effects, and further in some cases the performance or effectivity of the drug product may be compromised.

In the case of topical medications, the amount of drug product to treat the affliction may also approach amounts whereby unwanted or harmful side effects may occur and concentrations and amount of active pharmaceutical ingredients must be limited or controlled accordingly.

Accordingly, in order to prevent and reduce side effect of topical medications, the dosage of the drugs must be controlled very carefully. However, the efficacy of controlled dosage of drugs may also be dependent on the frequency of application of a drug product.

As the amount of active pharmaceutical ingredients in topical drug products is present in very small amounts in the topical medications, as the product may often to be worn away, for example, by the friction with clothes, contact with environment, washing hands, evaporation or the like, the need of frequent applications of the medications often causes inconvenience to the subject, and subject compliance with treatment regimens consequently may also adversely affect the efficacy of treatment.

Due to the side effects in many topically applied medicaments, a subject cannot apply too much medications at a time as a single dosage in order to seek to reduce the frequency of applications.

Accordingly, in order to improve the quality of life of the subject by both enhanced treatment and ease of application, a product method to reduce topically applied drugs products being worn away is needed.

### OBJECT OF THE INVENTION

It is an object of the present invention to provide a topical product and formulation for the treatment of dermal disorders which overcomes or at least partly ameliorates at least some deficiencies as associated with the prior art.

### SUMMARY OF THE INVENTION

In a first aspect, the present invention provides a conjugate comprising at least one calcineurin phosphatase inhibitor, and a nanodiamond.

In an embodiment, the at least one calcineurin phosphatase inhibitor may be adsorbed to the surface of the nanodiamond. The at least one calcineurin phosphatase inhibitor maybe adsorbed to the nanodiamond by way of a functional group on the surface of the nanodiamond.

In another embodiment, the at least one calcineurin phosphatase inhibitor may be bonded to the nanodiamond by way of covalent bonding functional group on the surface of the nanodiamond.

The functional group may be hydrogen (—H), carboxylate group (—COOH), amino group (—NH₂) or the like.

The nanodiamond is preferably sized so as to prevent permeation of the conjugate through the stratum corneum of a subject. The nanodiamond preferably has a size of greater than 50 nm.

The calcineurin phosphatase inhibitor may be tacrolimus (C₄₄H₆₉NO₁₂), or may be pimecrolimus (C₄₃H₆₈ClNO₁₁)

Preferably, the conjugate of acalcineurin phosphatase inhibitor and a nanodiamond is for use in the treatment of eczema.

In a second aspect, the present invention provides a topically administrable pharmaceutical composition for prevention or treatment of eczema of a subject, wherein the pharmaceutical composition comprises a plurality of conjugates of the first aspect and a pharmaceutical carrier.

The pharmaceutical carrier may be selected from the group including cream, an ointment, a gel, a foam, a lotion, a spray or the like.

The conjugate of a calcineurin phosphatase inhibitor and a nanodiamond inhibits the phosphatase activity of calcineurin.

The conjugation between the calcineurin phosphatase inhibitor and the nanodiamond is controllably rupturable, such that upon controlled rupture of said conjugation the phosphatase inhibitor is released so as to deliver said phosphatase inhibitor at a surgical site on the dermis of a subject.

The conjugation between the calcineurin phosphatase inhibitor and the nanodiamond is controllably rupturable upon application of ultra violet (UV), such that upon controlled rupture of said conjugation the phosphatase inhibitor is released so as to deliver said phosphatase inhibitor at a surgical site on the dermis of a subject.

The conjugation between the calcineurin phosphatase inhibitor and the nanodiamond is controllably rupturable upon application of ultra violet (UV) in range of from 320 to 360 nm such that the conjugation linkage is photolyzed, such that upon controlled rupture of said conjugation the phosphatase inhibitor is released so as to deliver said phosphatase inhibitor at a surgical site on the dermis of a subject.

In a third aspect, the present invention provides a method of preventing or treating a subject of a skin disorder, said method including the steps of (i) applying a pharmaceutical composition of the second aspect to a surgical site on the dermis of a subject, conjugation of the of at least one active pharmaceutical ingredient and said nanodiamond inhibits the release of the active pharmaceutical ingredient; and (ii) rupturing said conjugation between the active pharmaceutical ingredient and the nanodiamond, such that the active pharmaceutical ingredient is released so as to deliver said active pharmaceutical ingredient a surgical site on the dermis of a subject.

The conjugation between the active pharmaceutical ingredient and the nanodiamond may be controllably ruptured by applying of ultra violet (UV) to said pharmaceutical composition, upon such that upon controlled rupture of said conjugation the active pharmaceutical ingredient is released so as to deliver said active pharmaceutical ingredient at a surgical site on the dermis of a subject.

The conjugation between the active pharmaceutical ingredient and the nanodiamond may be controllably ruptured by applying ultra violet (UV) in range of from 320 to 360 nm such that the conjugation linkage is photolyzed, such that upon controlled rupture of said conjugation the active pharmaceutical ingredient is released so as to deliver said active pharmaceutical ingredient at a surgical site on the dermis of a subject.

The skin disorder is preferably eczema.

In a fourth aspect, the present invention provides for the use of a conjugate of the first aspect for the manufacture of a medicament for the prevention or treatment of eczema.

In a fifth aspect, the present invention provides conjugate of the first aspect for use in the prevention or treatment of eczema.

In a sixth aspect, the present invention provides a composition according to the second aspect for use in the prevention or treatment of eczema.

In a seventh aspect, the present invention provides a conjugate of the first aspect for use in a method for treating eczema.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1 depicts a schematic representation of the manner in which the invention is effected.

### DETAILED DESCRIPTION OF THE DRAWINGS

The present inventors have identified shortcomings of the prior art, and upon identification of the problems with the prior art, have provided a topical product and formulation which overcomes the problems of the prior art.

A relatively common dermal disorder suffered throughout the world, is atopic dermatitis, as known as atopic eczema or eczema, which is a very common dermal disease which patients suffer from red and itchy skin.

Other symptoms of eczema vary between individual subjects, which include dry skin, itching, red patches, raised bumps, thickened or scaly skin, and bleeding skin due to scratching.

The exact cause of eczema remains unknown, however it is thought to be related to genetic factors, as well as the environment a subject is exposed to as well as some degree of compromise or malfunction of the subject's immune system.

It is known that triggers factors of eczema are found to be irritants such as stress, nickel metal, cigarette smoke, chemicals used in daily cleaning products used cleaners, insects, soap products and even some skin care products.

Although the underlying cause of eczema may be unclear, there are ways in which to prevent the triggers of eczema.

One such approach is to prevent the triggering of eczema through inhibition. In the immune response with the irritants, calcineurin phosphatase is known as the beginning of a series of reaction, which can lead to the trigger of eczema.

Therefore, for eczema treatment, inhibitory drugs, such as tacrolimus (C₄₄H₆₉NO₁₂) and pimecrolimus (C₄₃H₆₈ClNO₁₁) are used to inhibit calcineurin phosphatase. For tacrolimus ointment, marked as Protopic, is available 0.03% ointment is for use on children aged 2 to 15 years while 0.03% or 0.1% ointment can be used by adults and children 16 years and older.

The most common observed and reported side effects of tacrolimus ointment as a result of skin application site include stinging, burning, or itching of the skin treated with the medication. Tacrolimus also is also reported to cause other side effects including acne, swollen or infected hair follicles, headache, increased sensitivity of the skin to hot or cold temperatures, flu-like symptoms such as the common cold and stuffy nose, skin tingling, upset stomach, muscle pain, enlarged lymph nodes, and skin infections like cold sores, chicken pox or shingles.

Tacrolimus is an immunosuppressant, and is a prescription drug with strictly prescribed dosage strength and regimes.

Pimecrolimus, currently marked as Elidel, is also an immunosuppressant like similarly to tacrolimus. Pimecrolimus has a similar mode of action to that of tacrolimus but is considered more selective.

Cream of 1% pimecrolimus is prescribed to treat mild to moderate eczema for adults and children age 2 years and older who do not have a weakened immune system.

Pimecrolimus is reported to cause common side effects such as burning or a feeling of warmth. Pimecrolimus can also cause other side effects such as headaches, common cold or stuffy nose, sore throat, cough, influenza, fever, swollen lymph nodes and viral infections like cold sores, chicken pox, shingles, or warts.

In accordance with the present invention, in order to control drug release for dermal diseases, the present invention is directed to a pharmaceutical product for topical administration for the prevention or treatment of such dermal diseases.

In particular, as an exemplary embodiment, the present invention provides a pharmaceutical product for topical administration for the prevention or treatment of the disorder eczema.

In accordance with the invention, a pharmaceutical composition including a conjugate comprising at least one calcineurin phosphatase inhibitor, and a nanodiamond is delivered to the skin of a patient. The conjugation can be subsequently ruptured for delivery of the calcineurin phosphatase inhibitor to the skin of the patient

For the skin disorder of eczema, nanodiamonds are attached with tacrolimus or pimecrolimus, so that the drugs can only stay on the surface of skin without deep skin penetration.

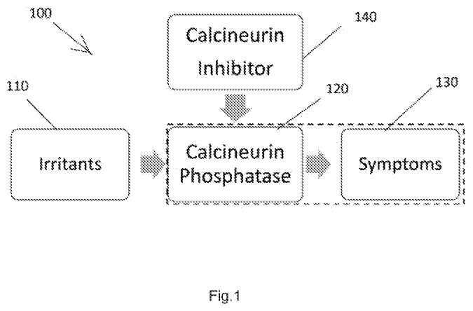

Referring to FIG. 1, there is provided a schematic representation of the implementation of the invention.

In the present invention 100, as shown, irritants 110 cause phosphatase activity of calcineurin 120 which results in the development of symptoms of eczema 130.

By applying a calcineurin phosphatase inhibitor 140 in accordance with the present invention by delivery via the calcineurin phosphatase inhibitor 140 by way of the calcineurin phosphatase inhibitor 140 being delivered being conjugated with nanodiamonds and released at the relevant site on the skin of a subject, in order to the development of symptoms of eczema 130.

In order to provide such a pharmaceutical product for prevention or treatment of eczema, nanodiamonds are conjugated with a drug known to be therapeutically active for dermal disease, to prevent drug diffusing too deeply into the skin tissue. Between nanodiamonds and the drugs, photochemical reactive linkages are provided for continuous or intermittent release of drugs.

Nanodiamonds (NDs) have been intensively studied for cytotoxicity within the art, and as diamond is carbon in nature with very little reactivity, it is considered to be highly biocompatible.

On the surface of nanodiamonds, there are various functional groups for terminations, such as hydrogen (—H), carboxylate group (—COOH), amino group (—NH₂) or the like. These functional groups provide the surface of nanodiamonds with the ability to bond with biomolecules or chemicals, such as in the present invention.

As should be understood, numerous biomolecules or chemicals may be attached to a single nanodiamond.

With a suitable combination of surface functional groups of nanodiamond, biomolecules or chemicals can also be made to adsorb on the surface physically without bonding.

The size of nanodiamonds can be engineered by various processes in the art, and such nanodiamonds are generally irregular in shape.

Different sizes of nanodiamonds have been demonstrated to have different levels of cell and tissue permeabilities. Studies have shown that carboxylated nanodiamonds with the size of approximately 50 nm cannot permeate through the stratum corneum, the outermost layer of the epidermis. On the other hand, the nanodiamonds may also stay in the hair follicles. This enables control of drug release physically by nanodiamonds.

For the skin disorder of eczema, nanodiamonds are attached with tacrolimus or pimecrolimus, so that the drugs can only stay on the surface of skin without deep skin penetration.

In order to perform controlled drug release in accordance with the present invention, the linkage between the drug and nanodiamonds can be photochemical reactive linkage. Upon exposure of ultraviolet (UV) light, in range of 320 to 360 nm, the linkage will be photolyzed.

Whilst the present invention in described with reference to conjugation of active ingredients having eczema related therapeutic properties, in other embodiments using such a controlled release mechanism, other or alternate active pharmaceutical ingredients may be conjugated with nanodiamonds as a controlled release conjugate mechanism.

According to the present invention, when the conjugated nanodiamond-drug complex is applied on skin, the nanodiamonds do not penetrate the skin and get into bloodstream; but rather, but rather remain on skin, possibly in the hair follicles, and function throughout the time.

Upon exposure of UV light, which may be obtained from sunlight, or potentially during phototherapy of eczema patient, the nanodiamond-drug complex will decompose and release the drug to the subject. The unbounded drug can therefore function by diffuse into deeper layers of skin as may be required for therapeutic activity.

In embodiments of the present invention, once a pharmaceutical composition is applied to the skin, the drug may then be continuously released under sunlight as long as it stays on the skin.

Advantageously, the controlled drug release of the active pharmaceutical ingredient as provided by the present invention for example activation under sunlight, can assist in avoiding overdose by mis-application of a pharmaceutical product, due its release mechanism.

Since the drug release can be controlled, patients only need to apply medication once for a prolonged period, which may also reduce unsafe levels in within the body and plod plasma, and provide a product which:
(i) is safer,
(ii) is more therapeutically effective,
(iii) reduces API loading to other parts of the body, and
(iv) allows for therapeutically useful dosage and treatment regimens by lessening loading.

As nanodiamonds can fit into hair follicles of a subject, and release medication from there under sunlight, a pharmaceutical product according to the present invention allows for topical delivery with increased ability to remain applied to the subject in the incidence of wiping or washing of skin.

1. A conjugate comprising:
(a) at least one calcineurin phosphatase inhibitor, and
(b) a nanodiamond.

2. The conjugate according to claim 1, wherein the at least one calcineurin phosphatase inhibitor is adsorbed to the surface of the nanodiamond.

3. The conjugate according to claim 1, wherein the at least one calcineurin phosphatase inhibitor is adsorbed to the nanodiamond by way of a functional group on the surface of the nanodiamond.

4. The conjugate according to claim 1, wherein the at least one calcineurin phosphatase inhibitor is bonded to the nanodiamond by way of covalent bonding functional group on the surface of the nanodiamond.

5. The conjugate according to claim 3, wherein the functional group is hydrogen (—H), carboxylate group (—COOH), amino group (—NH₂) or the like.

6. The conjugate according to claim 1, wherein the nanodiamond is sized so as to prevent permeation of the conjugate through the stratum corneum of a subject.

7. The conjugate according to claim 1, wherein the nanodiamond has a size of greater than 50 nm.

8. The conjugate according to claim 1, wherein the calcineurin phosphatase inhibitor is tacrolimus (C₄₄H₆₉NO₁₂).

9. The conjugate according to claim 1, wherein the calcineurin phosphatase inhibitor is pimecrolimus (C₄₃H₆₈ClNO₁₁).

10. A topically administrable pharmaceutical composition for prevention or treatment of eczema of a subject, wherein the pharmaceutical composition comprises a conjugate of claim 1 and a pharmaceutical carrier.

11. The pharmaceutical composition according to claim 10, wherein the pharmaceutical carrier is selected from the group including cream, an ointment, a gel, a foam, a lotion, a spray or the like.

12. The pharmaceutical composition according to claim 10, wherein the conjugate of a calcineurin phosphatase inhibitor and a nanodiamond inhibits the phosphatase activity of calcineurin

13. The pharmaceutical composition according to claim 10, wherein the conjugation between the calcineurin phosphatase inhibitor and the nanodiamond is controllably rupturable, such that upon controlled rupture of said conjugation the calcineurin phosphatase inhibitor is released so as to deliver said phosphatase inhibitor at a surgical site on the dermis of a subject.

14. The pharmaceutical composition according to claim 10, wherein the conjugation between the calcineurin phosphatase inhibitor and the nanodiamond is controllably rupturable upon application of ultra violet (UV), such that upon controlled rupture of said conjugation the phosphatase inhibitor is released so as to deliver said calcineurin phosphatase inhibitor at a surgical site on the dermis of a subject.

15. The pharmaceutical composition according to claim 10, wherein the conjugation between the calcineurin phosphatase inhibitor and the nanodiamond is controllably rupturable upon application of ultra violet (UV) in range of from 320 to 360 nm such that the conjugation linkage is photolyzed, such that upon controlled rupture of said conjugation the calcineurin phosphatase inhibitor is released so as to deliver said phosphatase inhibitor at a surgical site on the dermis of a subject.

16. A method of preventing or treating a subject of a skin disorder, said method including the steps of:
(i) applying the pharmaceutical composition of claim 10 to a surgical site on the dermis of a subject, conjugation of the of at least one active pharmaceutical ingredient and said nanodiamond inhibits the release of the active pharmaceutical ingredient; and
(ii) rupturing said conjugation between the active pharmaceutical ingredient and the nanodiamond, such that the active pharmaceutical ingredient is released so as to deliver said active pharmaceutical ingredient a surgical site on the dermis of a subject.

17. The method according to claim 16, wherein the conjugation between the active pharmaceutical ingredient and the nanodiamond is controllably ruptured by applying of ultra violet (UV) to said pharmaceutical composition, upon such that upon controlled rupture of said conjugation the active pharmaceutical ingredient is released so as to deliver said active pharmaceutical ingredient at a surgical site on the dermis of a subject.

18. The method according to claim 16, wherein the conjugation between the active pharmaceutical ingredient and the nanodiamond is controllably ruptured by applying ultra violet (UV) in range of from 320 to 360 nm such that the conjugation linkage is photolyzed, such that upon controlled rupture of said conjugation the active pharmaceutical ingredient is released so as to deliver said active pharmaceutical ingredient at a surgical site on the dermis of a subject.

19. The method according to claim 16, wherein said skin disorder is eczema.

20. The use of a conjugate of claim 1, for the manufacture of a medicament for the prevention or treatment of eczema.

21. The conjugate of claim 1, for use in the prevention or treatment of eczema.

22. The composition according to claim 10, for use in the prevention or treatment of eczema.

23. The conjugate of claim 1 for use in a method for treating eczema.

24. The conjugate according to claim 4, wherein the functional group is hydrogen (—H), carboxylate group (—COOH), amino group (—NH₂) or the like.
